(19) United States
(12) Patent Application Publication
(10) Pub. No.: US 2024/0368635 A1
(43) Pub. Date: Nov. 7, 2024

(54) METHOD AND PLANT FOR PROCESSING BIOLOGICAL SUBSTRATE

(71) Applicant: Schmack Biogas Service GmbH, Schwandorf (DE)

(72) Inventor: Edgar HÖSLER, Schwandorf (DE)

(21) Appl. No.: 18/686,671

(22) PCT Filed: Sep. 1, 2021
(86) PCT No.: PCT/EP2021/074157

§ 371 Date: Feb. 26, 2024

(51) Int. Cl. C12P 5/02; C05F 17/60; C12M 1/00; C12M 1/107; C12M 1/26

(52) U.S. Cl. CPC C12P 5/023; C05F 17/60; C12M 21/04; C12M 23/58; C12M 29/14; C12M 33/14; C12M 47/10

(57) ABSTRACT

The invention relates to a method for processing biological substrate, comprising the steps of: a) introducing the biological substrate into an apparatus for dry fermentation, b) carrying out dry fermentation to recover methane, forming fermentation residue, c) transferring the fermentation residue obtained in step b) to an apparatus for solid-liquid separation, d) carrying out solid-liquid separation of the fermentation residue, forming a fermentation residue solid phase and a fermentation residue raw liquid phase, e) transferring the fermentation residue raw liquid phase obtained in step d) to an apparatus for separating solids, f) carrying out separation of solids from the fermentation residue raw liquid phase, recovering a fermentation residue liquid phase, g) introducing the fermentation residue liquid phase obtained in step f) into an apparatus for wet fermentation, h) carrying out wet fermentation to recover methane, forming liquid fermentation residue, i) returning at least some of the liquid fermentation residue formed in step h) as inoculum to the apparatus for dry fermentation.

### TECHNICAL FIELD

The invention relates to a method and a plant for processing biological substrate.

### DESCRIPTION OF THE RELATED ART

Biogas plants produce methane through a microbial degradation process of organic substances. The biogas is produced in a multi-stage process of fermentation or digestion through the activity of anaerobic microorganisms, i.e. in the absence of atmospheric oxygen. Depending on the type of biomass to be fermented, a distinction is made between dry and wet fermentation.

Dry fermentation uses fibrous, solid biological substrates such as manure, green waste, biowaste from separate collection and organic fractions from residual waste. The biological substrates used in dry fermentation generally contain a water content of up to 70% and a dry matter content of more than 15%. The designation of fermentation as “dry” therefore only serves to differentiate it from wet fermentation and should not be understood to mean that dry biomass is actually used. The biological substrate always contains solid impurities such as sand, stones, wood, glass or metal.

Plug-flow fermenters are often used to carry out dry fermentation. Fermentation in the plug-flow fermenter leads to a breakdown of the fatty acids in the fermentation substrate as well as to a general breakdown of the dry organic matter present in the biowaste. In order to ensure sufficient fermentation to maximise the gas yield, relatively long residence times (usually longer than 20 days) of the substrate in the plug-flow fermenter must be observed. This leads to high investment costs, as the size of the plug-flow fermenter must be designed according to the minimum residence time. The optimised economic efficiency of the plant due to the high gas yield is therefore reduced by the high investment costs required.

For dry fermentation, an inoculum must be provided that contains sufficient microbiologically active organisms to enable the necessary microbial processes, i.e. the anaerobic degradation that takes place in the fermenter. As a rule, this inoculum is a liquid fraction from the fermentation residue that leaves the plug-flow fermenter and is returned for mashing of the biological substrate.

In order to prevent a possible over-acidification of the fermenter due to an excessively high concentration of organic fatty acids in the material returned as inoculum, the fermentation substrate must be digested as far as possible, which also requires long residence times in the dry fermenter.

With correspondingly long residence times in the dry fermenter, the organic substrate is degraded to the greatest possible extent, which has at least two negative effects on the process. On the one hand, the solid part of the fermentation residue after dry fermentation is usually fed to an aerobic treatment with an intentionally short residence time, namely composting or post-rotting, in order to achieve a further reduction and stabilisation as well as a hygienisation of the biowaste. To ensure this, a minimum temperature must be maintained during composting. However, this is difficult to achieve with largely digested biowaste, which only contains a small amount of organic matter, so that fresh organic material often has to be added for composting. This means that fresh substrate is consumed that has not previously undergone dry fermentation and therefore does not contribute to the methane yield of the plant, which reduces the economic efficiency of the plant.

On the other hand, a disadvantage of extensive digestion is that more inert, non-degradable substances such as bones or metals are deposited towards the outlet of the plug-flow fermenter, i.e. in a region in which the dry matter content becomes increasingly lower. Such deposits occur because the capacity of the fermenter suspension for inert substances decreases more and more due to the extensive digestion. The deposits can lead to increased wear and even failure of the fermenter's agitator equipment.

A dry fermentation plant is described, for example, in DE 10 2013 213 258 A1. There, in a process for producing a liquid fertiliser in a dry fermentation plant, a liquid percolate is produced which is collected in a percolate tank. The percolate is fed back into the dry fermentation process as inoculum. After hygienisation, the liquid fermentation residue is spread from the percolate tank as fertiliser.

Dry fermenters are also used in combination with wet fermentation plants in which substrates with a dry matter content of less than 15% are used. In such plants, the fermentation residues obtained from dry fermentation are fed to the wet fermenter as a substrate.

WO 2019/086649 A1, for example, describes an annular plug-flow fermenter that surrounds a wet fermenter. The fermentation residue is fed from the outlet of the plug-flow fermenter into the wet fermenter as a substrate. WO 2019/086649 A1 thus discloses a method for processing biological substrate comprising the steps of a) introducing the biological substrate into a dry fermentation apparatus, b) carrying out a dry fermentation to obtain methane while forming a fermentation residue, c) introducing the fermentation residue obtained in step b) into an apparatus for wet fermentation, d) carrying out a wet fermentation to obtain methane with formation of a liquid fermentation residue.

The disadvantages of the solutions described in the prior art lie in particular in the relatively large dimensions of the dry fermenters due to the long minimum residence time of the substrate, in the increased wear of the agitator equipment of the fermenter caused by deposits of inert material, and in the additional consumption of biowaste as an admixture in the composting of the solid fermentation residue portion of the dry fermentation.

### SUMMARY

The object of the invention, as characterised in the claims, is to provide an economically optimised method for processing biological substrate which provides an increased yield of methane with an increased throughput of substrate per time.

According to the invention, this object is achieved by the method for processing biological substrate according to claim 1 and by the plant according to claim 12. Further advantageous details, aspects and embodiments of the present invention can be found in the dependent claims, the description, the FIGURE and the examples.

The present invention provides a method for processing biological substrate comprising the following steps:
a) introducing the biological substrate into a dry fermentation apparatus,
b) carrying out dry fermentation to obtain methane with formation of a fermentation residue,
c) transferring the fermentation residue obtained in step b) to an apparatus for solid/liquid separation,
d) carrying out a solid/liquid separation of the fermentation residue to form a fermentation residue solid phase and a fermentation residue liquid crude phase,
e) transferring the fermentation residue liquid crude phase obtained in step d) to an apparatus for separating solids,
f) carrying out a separation of solids from the fermentation residue liquid crude phase to obtain a fermentation residue liquid phase,
g) introducing the fermentation residue liquid phase obtained in step f) into an apparatus for wet fermentation,
h) carrying out a wet fermentation to obtain methane with formation of a liquid fermentation residue,
i) returning at least part of the liquid fermentation residue formed in step h) as inoculum to the dry fermentation apparatus.

The particular advantage of the method according to the invention lies in the reduction of the residence time of the substrate in the dry fermentation by at least 20% and up to 40%. The method according to the invention is suitable both for the expansion of an existing plant and for the construction of a new plant. In the case of existing plants, the wet fermenter must generally be added to the existing dry fermentation plant in a suitable size. By shortening the residence time in the dry fermentation process, a percentage increase in the throughput of biological substrate per time can be achieved with the same size of fermenter, so that a small plant achieves a higher total gas production. When building a new combined dry and wet fermentation plant, for example, a smaller dry fermenter can be built for the same amount of waste, thus saving costs.

By reducing the residence time of the substrate in the dry fermenter, an economic optimisation of the process is also achieved. On the one hand, higher throughputs of biological substrate are achieved in the dry fermenter as a percentage of the reduction in residence time. In addition, higher gas yields are achieved because the liquid phase of dry fermentation is completely digested in combination with wet fermentation.

The shortened residence time also makes it possible to feed (compared to conventional plants) a higher proportion of organic material as a fermentation residue solid phase into a downstream rotting process after carrying out a solid/liquid separation of the fermentation residue from the dry fermentation. In this case, the rotting process largely manages without the addition of fresh biowaste. Nevertheless, the rotting can be operated at a sufficiently high temperature and thus with good hygienisation and extensive water reduction, so that the composted material subsequently meets the requirements of the German Compost Quality Association.

As already mentioned, one disadvantage of extensive digestion of the substrate in the dry fermenter is that more inert, non-degradable substances such as bones or metals are deposited towards the outlet of the fermenter, i.e. in a region in which the dry matter content becomes increasingly lower. Such deposits occur because the capacity of the fermenter suspension for inert substances decreases more and more due to the extensive digestion. The deposits can lead to increased wear and even failure of the fermenter's agitator equipment. The reduction in the residence time of the substrate in the dry fermenter and the resulting lower degradation of organic material result in a higher proportion of organic matter and thus a higher carrying capacity of the fermenter suspension for inert impurities. The problem of the deposition of these impurities towards the outlet of the fermenter thus becomes less relevant because the settling in the dry fermenter is reduced.

These undeposited inert impurities then form part of the fermentation residue leaving the dry fermenter. As a significant proportion of this fermentation residue is to be used as a substrate for wet fermentation, these impurities pose a problem. In order to separate the impurities from the fermentation residue of the dry fermentation before the wet fermentation, it is provided according to the invention to first transfer the fermentation residue into an apparatus for solid/liquid separation after the dry fermentation has been carried out, to carry out a solid/liquid separation of the fermentation residue with formation of a fermentation residue solid phase and a fermentation residue liquid crude phase, and then to carry out a further separation step. For this purpose, the fermentation residue liquid crude phase is transferred to an apparatus for separating solids, and solids are separated from the fermentation residue liquid crude phase in this apparatus to obtain a fermentation residue liquid phase. This fermentation residue liquid phase is substantially free of impurities and can subsequently be introduced as a substrate into an apparatus for wet fermentation.

As already mentioned, an inoculum must be provided for dry fermentation that contains sufficient microbiologically active organisms to enable the necessary microbial processes, i.e. the anaerobic degradation that takes place in the fermenter. As a rule, a liquid fraction from the fermentation residue of the dry fermentation is used as inoculum and recycled for mashing the biological substrate. Due to the reduced residence time of the substrate in the dry fermentation according to the present invention, the fermentation residue leaving the dry fermenter is not sufficiently digested and therefore has too high a concentration of organic fatty acids. If used as inoculum, the fermenter would become over-acidified.

According to the invention, wet fermentation is therefore carried out downstream of dry fermentation to produce methane. The methane is produced with the formation of a liquid fermentation residue. In this process, the material fed to the wet fermentation is sufficiently digested so that the liquid fermentation residue can be fed to the dry fermentation process as inoculum without the risk of over-acidification. According to the invention, it is therefore envisaged that at least part of the liquid fermentation residue formed during wet fermentation is fed as inoculum back into the dry fermentation apparatus.

When the method according to the invention is carried out, the dry fermenter and the wet fermenter are therefore used together to process the biological substrate, which results in particularly high gas yields. The gas yields increase sharply, as the combination of dry and wet fermentation means that the starting material is completely digested or hygienised in the method according to the invention, but, due to the shorter residence time in the dry fermenter, more fermentation substrate can be introduced overall compared to conventional plants.

Preferably, as step i), the step
i) returning a first portion of the liquid fermentation residue formed in step h) as inoculum to the dry fermentation apparatus and transferring a second portion of the liquid fermentation residue formed in step h) to an apparatus for hygienisation is carried out, and, after step i), the step
j) hygienising the second portion of the liquid fermentation residue formed in step h) to form an agriculturally usable biological material is carried out. With this preferred embodiment, excess material from the wet fermentation, which is not returned to the dry fermentation apparatus as inoculum, can be used as agricultural fertiliser on agricultural land after hygienisation. As a rule, the transfer to agriculture takes place after prior intermediate storage of the agriculturally utilisable biological material.

Particularly preferably, in step j) the hygienisation takes place at a temperature between 65° C. and 75° C., especially preferably at a temperature of 70° C. Also preferably, in step j) the hygienisation takes place with a residence time of the liquid fermentation residue of between 45 min and 75 min, in particular preferably with a residence time of 60 min. With the preferred temperatures and residence times mentioned, a particularly optimal hygienisation is achieved, whereby all liquid fermentation residue formed in step h), which is not returned as inoculum to the dry fermentation apparatus, can be used as agricultural fertiliser on agricultural land.

According to a further preferred embodiment of the present invention, after step d), the steps
d₀) transferring the fermentation residue solid phase obtained in step d) to an apparatus for composting biological material
and
d₁) carrying out composting of the fermentation residue solid phase obtained in step d) in the apparatus for composting biological material are carried out. Particularly preferably, composting is carried out in step d₁) without admixing additional, untreated biological substrate. Due to the shortened residence time in the dry fermenter, a higher proportion of organic material compared to conventional plants can be fed to a downstream rotting process as a fermentation residue solid phase after a solid/liquid separation of the fermentation residue from the dry fermentation has been carried out. As a rule, this rotting process does not require any admixture of fresh biowaste. Nevertheless, rotting is achieved at a sufficiently high temperature and thus with good hygienisation and extensive water reduction, so that the composted material subsequently meets the specifications of the German Compost Quality Association. The process according to the preferred embodiment described leads to savings in operating costs and construction costs.

Preferably, the separation of solids from the fermentation residue liquid crude phase obtained by the solid/liquid separation of the fermentation residue is carried out in step f) by density separation to obtain a fermentation residue liquid phase. Particularly preferably, the density separation is carried out using a press, a grit separator, a decanter, a sieve or a settling tank.

According to a further preferred embodiment, the fermentation residue liquid phase introduced into the apparatus for wet fermentation in step g) has a dry matter content of less than 15%. With a dry matter content of less than 15%, the wet fermentation proceeds with a particularly high yield of methane.

Preferably, the wet fermentation carried out in step h) is carried out with the admixture of additional biological substrate. If the wet fermenter is dimensioned accordingly, additional external liquid input flows can also be processed and subsequently utilised. This results in a broader and more flexible utilisation of the entire fermentation plant of dry and wet fermentation. The additional biological substrate admixed in step h) is preferably liquid manure, catering waste, food scraps, market waste or mixtures of these substrates.

As already mentioned, the method according to the invention makes it possible to shorten the residence time of the biological substrate in the dry fermentation apparatus compared to conventional plants and methods. A particularly preferred embodiment of the present invention provides for carrying out the dry fermentation in step b) to obtain methane with a residence time of the biological substrate in the dry fermentation apparatus of 10 to 19 days, particularly preferably with a residence time of 12 to 17 days.

The solids separated from the fermentation residue liquid crude phase in step f) are usually stones, sand, glass and/or pieces of metal.

The biological substrate introduced into the dry fermentation apparatus in step a) preferably has a dry matter content of between 15% and 60%, particularly preferably between 25% and 50%. Particularly high gas yields are achieved when using biological substrates with the aforementioned dry matter content.

The present invention also comprises a plant for processing biological substrate comprising at least one dry fermentation apparatus, at least one apparatus for solid/liquid separation, at least one apparatus for separating solids, at least one apparatus for wet fermentation, and at least one apparatus for returning liquid fermentation residue as inoculum to the dry fermentation apparatus.

Preferably, an apparatus for the hygienisation of liquid fermentation residue is additionally provided. As already mentioned, at least some of the liquid fermentation residue produced during wet fermentation is returned to the dry fermenter as inoculum. In the event that not all of the liquid fermentation residue is required as inoculum, an apparatus for the hygienisation of liquid fermentation residue is provided, with the aid of which the excess liquid fermentation residue can be hygienised. As a result of this hygienisation, all liquid fermentation residue formed during wet fermentation that is not returned to the dry fermentation apparatus as inoculum can be used as agricultural fertiliser on agricultural land.

The dry fermentation apparatus is preferably a plug-flow fermenter. A plug-flow fermenter enables continuous processing of substrates by dry fermentation. The substrate is conveyed here as a “plug” through the horizontal fermenter by means of large hydraulic piston pumps.

According to a particularly preferred embodiment, the apparatus for solid/liquid separation is a press screw separator. With the aid of the press screw separator, a solid/liquid separation of the fermentation residue is carried out with the formation of a fermentation residue solid phase enriched with solids and a fermentation residue liquid crude phase depleted of solids. This separates the coarse solids contained in the fermentation residue, such as plant fibres or husks.

The press-screw separator in this case conveys the fermentation residue to be dewatered in a cylindrical sieve drum by means of a screw against the discharge flaps weighted with weights. The coarse-grained fermentation residue components are pressed off at the discharge flaps and form a solids cake. The liquid passes through the gaps of the sieve drum into the jacket space and drains off there. The fermentation residue liquid crude phase preferably flows freely into a collecting tank. This preferably serves as a storage tank for the subsequent method step.

The fermentation residue solid phase precipitates at the discharge flap and is preferably transferred to an apparatus for composting biological material. According to a preferred embodiment, the plant for processing biological substrate additionally has an apparatus for composting biological material. In a particularly preferred embodiment, the apparatus for composting biological material is a post-rotting apparatus. The compost formed in the apparatus for composting biological material can, after a fine treatment process in which a fine grain <15 mm, preferably <11 mm, is used, be transported to fields or further dried and/or otherwise used.

The apparatus for separating solids is preferably a press, a grit separator, a decanter, a sieve or a settling tank. These apparatuses are particularly suitable for separating out the impurities frequently contained in the biological substrate, such as stones, sand, glass and/or pieces of metal.

The advantages associated with the plant for processing biological substrate according to the invention and the described preferred embodiments of this plant have already been described in conjunction with the embodiments of the method according to the invention.

Reference is therefore made to the corresponding text passages describing the advantages of the method according to the invention. These passages also reflect the advantages of the plant according to the invention.

### DETAILED DESCRIPTION

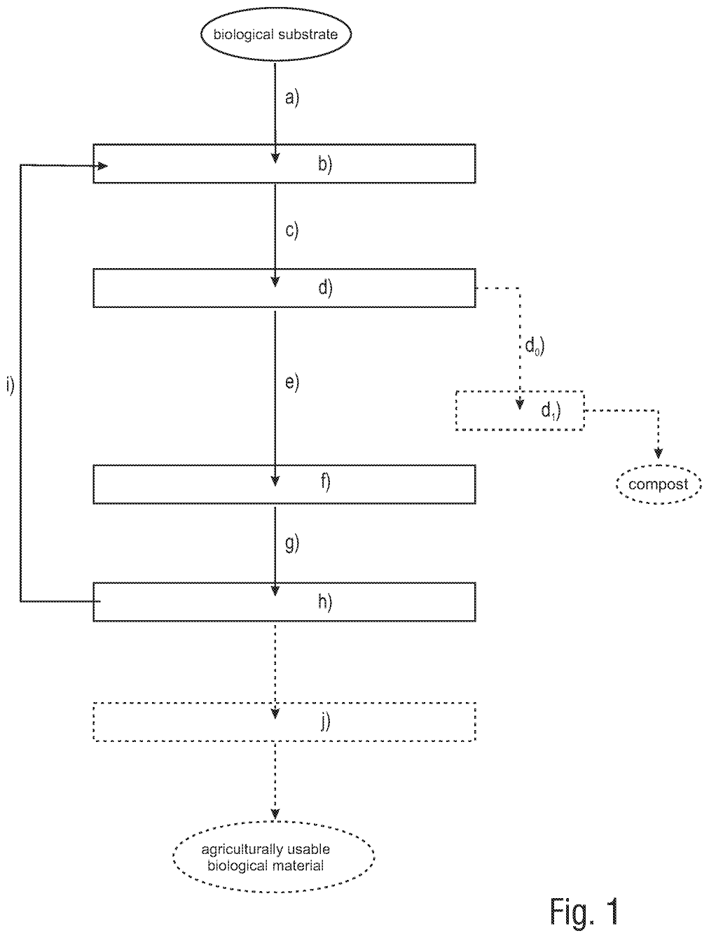

The invention will be explained in greater detail below with reference to an exemplary embodiment in conjunction with FIG. 1. However, it is expressly pointed out that the invention is not intended to be limited to the example given.

FIG. 1 uses a flow chart to show the sequence of the method according to the invention for processing biological substrate. All method steps shown with solid lines are part of the method according to the invention. The method steps shown with dashed lines are preferred embodiments.

The biological substrate is introduced into a dry fermentation apparatus in step a). The dry fermentation apparatus is preferably a plug-flow fermenter.

Step b) of the method according to the invention consists of carrying out a dry fermentation in the dry fermentation apparatus, wherein methane is recovered with the formation of a fermentation residue.

According to step c), the fermentation residue obtained in step b) is transferred to an apparatus for solid/liquid separation. This apparatus for solid/liquid separation is preferably a press-screw separator.

In the apparatus for solid/liquid separation, i.e. according to a preferred embodiment in the press-screw separator, a solid/liquid separation of the fermentation residue is carried out in step d), forming a fermentation residue solid phase and a fermentation residue liquid crude phase. The fermentation residue obtained from the dry fermentation in step b) is thus divided into a fermentation residue solid phase and a fermentation residue liquid crude phase.

According to a preferred embodiment, the fermentation residue solid phase obtained in step d) is transferred in step d₀) to an apparatus for composting biological material and, in step d₁), the fermentation residue solid phase obtained in step d) is composted in the apparatus for composting biological material. This composting in step d₁) is preferably carried out without admixing additional, untreated biological substrate. The shortened residence time of the substrate in the dry fermenter means that, compared to conventional plants, a higher proportion of organic material can be fed to a downstream rotting process as a fermentation residue solid phase after a solid/liquid separation of the fermentation residue from the dry fermentation has been carried out. This rotting process then generally does not require the admixture of fresh biowaste. Nevertheless, rotting is achieved at a sufficiently high temperature and thus with good hygienisation and extensive water reduction, so that the composted material then meets the specifications of the German Compost Quality Association.

The fermentation residue liquid crude phase obtained in step d) is transferred in step e) of the method according to the invention to an apparatus for separating solids. The apparatus for separating solids is preferably a press, a grit separator, a decanter, a sieve or a settling tank. In this apparatus for separating solids, solids are then separated from the fermentation residue liquid crude phase in step f) to obtain a fermentation residue liquid phase.

In step g) of the method according to the invention, the fermentation residue liquid phase obtained in step f) is introduced into an apparatus for wet fermentation. In the wet fermenter, wet fermentation is carried out in accordance with step h) to obtain methane, with formation of a liquid fermentation residue.

According to step i), at least part of the liquid fermentation residue formed in step h) is returned as inoculum to the dry fermentation apparatus.

In the event that not all of the liquid fermentation residue is required as inoculum, an apparatus for the hygienisation of liquid fermentation residue is provided, with the aid of which the excess liquid fermentation residue can be hygienised. In this case, the step
i) returning a first portion of the liquid fermentation residue formed in step h) as inoculum to the dry fermentation apparatus and transferring a second portion of the liquid fermentation residue formed in step h) to an apparatus for hygienisation is preferably carried out as step i), and, after step i), the step
j) hygienising the second portion of the liquid fermentation residue formed in step h) to form an agriculturally usable biological material is carried out. As a result of this hygienisation, the liquid fermentation residue formed during wet fermentation, which is not returned as inoculum to the dry fermentation apparatus, can be used as agricultural fertiliser on agricultural land.

The hygienisation in step j) is preferably carried out at a temperature of 70° C. with a residence time of the liquid fermentation residue of 60 min. Under these conditions, a particularly optimal hygienisation is achieved, whereby all liquid fermentation residue formed in step h), which is not returned to the dry fermentation apparatus as inoculum, can be used as agricultural fertiliser on agricultural land.

When using the method according to the invention, very high gas yields are achieved, as the combination of dry and wet fermentation, which is made possible by the intermediate separation steps, means that the starting material is completely digested or hygienised, but, due to the shorter residence time in the dry fermenter, more fermentation substrate can be introduced overall compared to conventional plants.

1. A method for processing biological substrate comprising the following steps:
a) introducing the biological substrate into a dry fermentation apparatus,
b) carrying out dry fermentation to obtain methane with formation of a fermentation residue,
c) transferring the fermentation residue obtained in step b) to an apparatus for solid/liquid separation,
d) carrying out a solid/liquid separation of the fermentation residue to form a fermentation residue solid phase and a fermentation residue liquid crude phase,
e) transferring the fermentation residue liquid crude phase obtained in step d) to an apparatus for separating solids,
f) carrying out a separation of solids from the fermentation residue liquid crude phase to obtain a fermentation residue liquid phase,
g) introducing the fermentation residue liquid phase obtained in step f) into an apparatus for wet fermentation,
h) carrying out a wet fermentation to obtain methane with formation of a liquid fermentation residue,
i) returning at least part of the liquid fermentation residue formed in step h) as inoculum to the dry fermentation apparatus.

2. The method according to claim 1, wherein, as step i), the step
i) returning a first portion of the liquid fermentation residue formed in step h) as inoculum to the dry fermentation apparatus and transferring a second portion of the liquid fermentation residue formed in step h) to an apparatus for hygienisation is carried out, and, after step i), the step
j) hygienising the second portion of the liquid fermentation residue formed in step h) with formation of an agriculturally usable biological material,
is carried out.

3. The method according to claim 2, wherein in step j) the hygienisation takes place at a temperature between 65° C. and 75° C., preferably at a temperature of 70° C.

4. The method according to claim 2, wherein in step j) the hygienisation takes place with a residence time of the liquid fermentation residue of between 45 min and 75 min, preferably with a residence time of 60 min.

5. The method according to claim 1, wherein, after step d), the steps
d₀) transferring the fermentation residue solid phase obtained in step d) to an apparatus for composting biological material
and
d₁) carrying out composting of the fermentation residue solid phase obtained in step d) in the apparatus for composting biological material are carried out.

6. The method according to claim 5, wherein in step d₁) the composting is carried out without admixing additional, untreated biological substrate.

7. The method according to claim 1, wherein the separation of solids from the fermentation residue liquid crude phase to obtain a fermentation residue liquid phase in step f) is carried out by density separation, in particular by density separation using a press, a grit separator, a decanter, a sieve or a settling tank.

8. The method according to claim 1, wherein the fermentation residue liquid phase introduced into the apparatus for wet fermentation in step g) has a dry matter content of less than 15%.

9. The method according to claim 1, wherein the wet fermentation carried out in step h) is carried out with admixture of additional biological substrate.

10. The method according to claim 9, wherein the additional biological substrate admixed in step h) is manure, catering waste, food scraps, market waste or mixtures of these substrates.

11. The method according to claim 1, wherein in step b) the dry fermentation to obtain methane is carried out with a residence time of the biological substrate in the dry fermentation apparatus of 10 to 19 days, preferably with a residence time of 12 to 17 days.

12. The method according to claim 1, wherein the solids separated from the fermentation residue liquid crude phase in step f) are stones, sand, glass and/or pieces of metal.

13. The method according to claim 1, wherein the biological substrate introduced into the dry fermentation apparatus in step a) has a dry matter content of between 15% and 60%, preferably between 25% and 50%.

14. A plant for processing biological substrate comprising
at least one dry fermentation apparatus,
at least one apparatus for solid/liquid separation,
at least one apparatus for separating solids,
at least one apparatus for wet fermentation, and
at least one apparatus for returning liquid fermentation residue as inoculum to the dry fermentation apparatus.

15. The plant according to claim 14, wherein an apparatus for the hygienisation of liquid fermentation residue is additionally provided.

16. The plant according to claim 14, wherein an apparatus for composting biological material is additionally provided.

17. The plant according to claim 14, wherein the dry fermentation apparatus is a plug-flow fermenter.

18. The plant according to claim 14, wherein the solid/liquid separation apparatus is a press-screw separator.

19. The plant according to claim 14, wherein the apparatus for separating solids is a press, a grit separator, a decanter, a sieve or a settling tank.

20. The plant according to claim 16, wherein the apparatus for composting biological material is a post-rotting apparatus.
